(19) United States
(12) Plant Patent Application Publication
(10) Pub. No.: US 2012/0204301 P1
(43) Pub. Date: Aug. 9, 2012

(54) Gerbera Plant Named 'UFGE 4141'

(50) ;

(76) Inventors: Zhanao Deng, Riverview, FL (US); Brent K. Harbaugh, (US)

(21) Appl. No.: 12/931,572

(22) Filed: Feb. 4, 2011

(51) Int. Cl. A01H 5/00

(52) U.S. Cl. PLT/357

(57) ABSTRACT

A new Gerbera plant particularly distinguished by having a novel combination of medium lengths of peduncles, semi-double orange-red flower color, a moderate level of resistance to powdery mildew, and demonstrated potential to produce attractive plants in large (≧20-cm in diameter) containers, is disclosed.

### GENUS AND SPECIES

Gerbera hybrida

### VARIETY DENOMINATION

‘UFGE 4141’

### BACKGROUND OF THE NEW PLANT

The invention relates to a new and distinct variety of Gerbera plant named ‘UFGE 4141’. ‘UFGE 4141’ originated from cross made between ‘UFGE 5-23’ (unpatented) and ‘UFGE 4-8’ (unpatented), during 2003 at Bradenton, Fla. ‘UFGE 4141’ was selected by the inventors from the progeny of the stated parentage in summer 2004 at Bradenton, Fla. The first asexual reproduction of ‘UFGE 4141’ was accomplished when crown division was done in fall 2004 in Bradenton, Fla. Plants of ‘UFGE 4141’ have been asexually propagated by crown division and/or tissue culture for more than four generations. Asexually propagated plants of ‘UFGE 4141’ have remained true to the original selected plant, and all characteristics of the plant have been transmitted and retained through four successive asexual vegetative generations.

Plant Breeder's Rights for this cultivar have not been applied for. ‘UFGE 4141’ has not been made publicly available more than one year prior to the filing of this application.

### SUMMARY OF THE INVENTION

The new and distinct variety of Gerbera plant is characterized by a novel combination of medium lengths of peduncles, semi-double orange-red flowers, a moderate level of resistance to powdery mildew, and demonstrated potential to produce attractive plants in large (≧20-cm in diameter) containers. ‘UFGE 4141’ has not been observed under all possible environmental conditions. Its phenotype may vary significantly with variations in environment such as light intensity, temperature, and day length. The following are the most outstanding and distinguishing characteristics of this new cultivar when grown in Wimauma, Fla. under normal horticultural practices in greenhouse conditions which closely approximate those generally used in commercial practice.

1. Peduncle length of approximately 47 cm;

2. Semi-double flower type;

3. Orange-red (RHS 33B) ray florets;

4. Yellow-green (RHS 150B) color discs before opening of disc florets;

5. Orange-red (RHS 33A) perianth lobe of disc florets;

6. Flower head diameter of approximately 120 mm; and

7. Moderate resistance to powdery mildew.

### DESCRIPTION OF THE PHOTOGRAPHS

This new Gerbera plant is illustrated by the accompanying photographs which show the plant's form, flower head, and foliage. The colors shown are as true as can be reasonably obtained by conventional photographic procedures. The photographs are of a plant approximately 5 months old which was produced from one tissue culture liner and is potted in a 2.7-L container. FIG. 3 is of a leaf removed from the same 5 month old plant.

FIG. 1 shows the overall plant habit including flowers and foliage and is taken from a side perspective view.

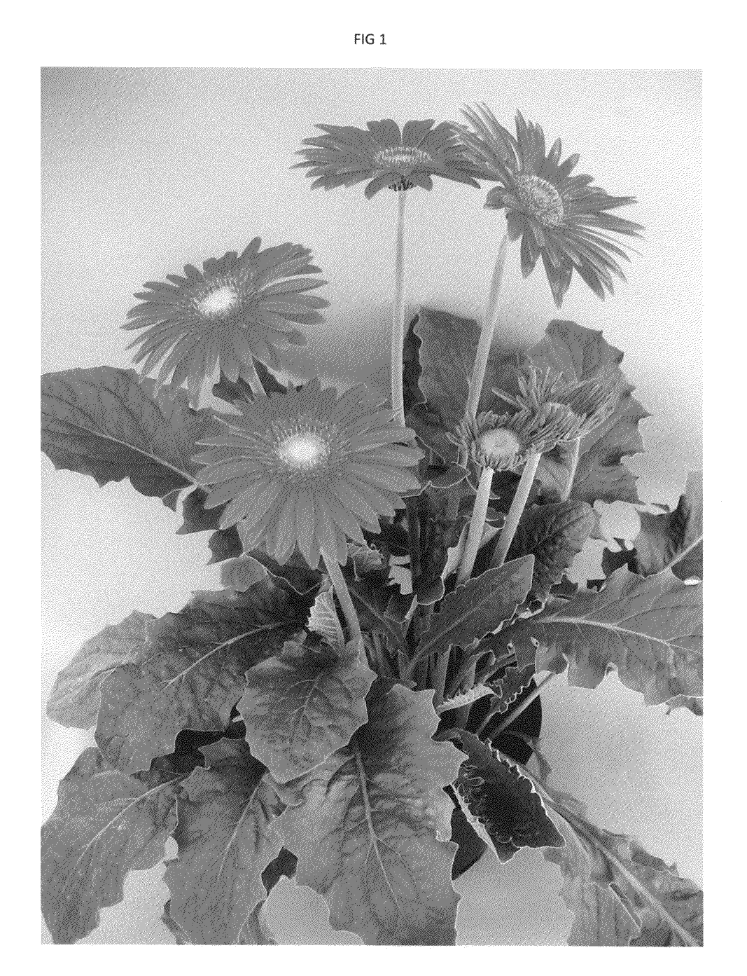

FIG. 2 shows a close-up of the flower head.

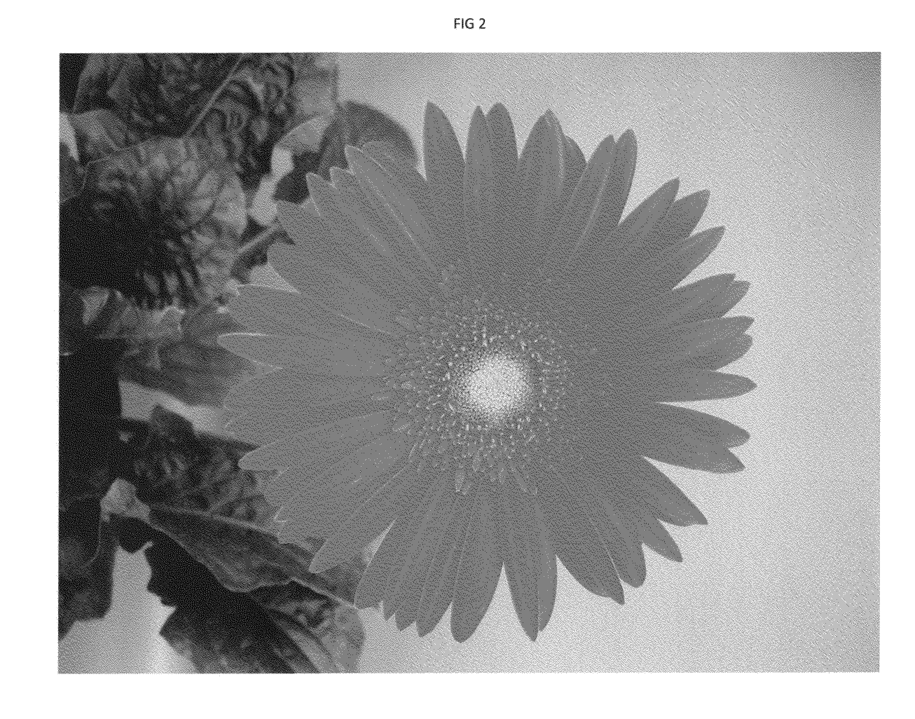

FIG. 3 shows a close-up of a typical leaf.

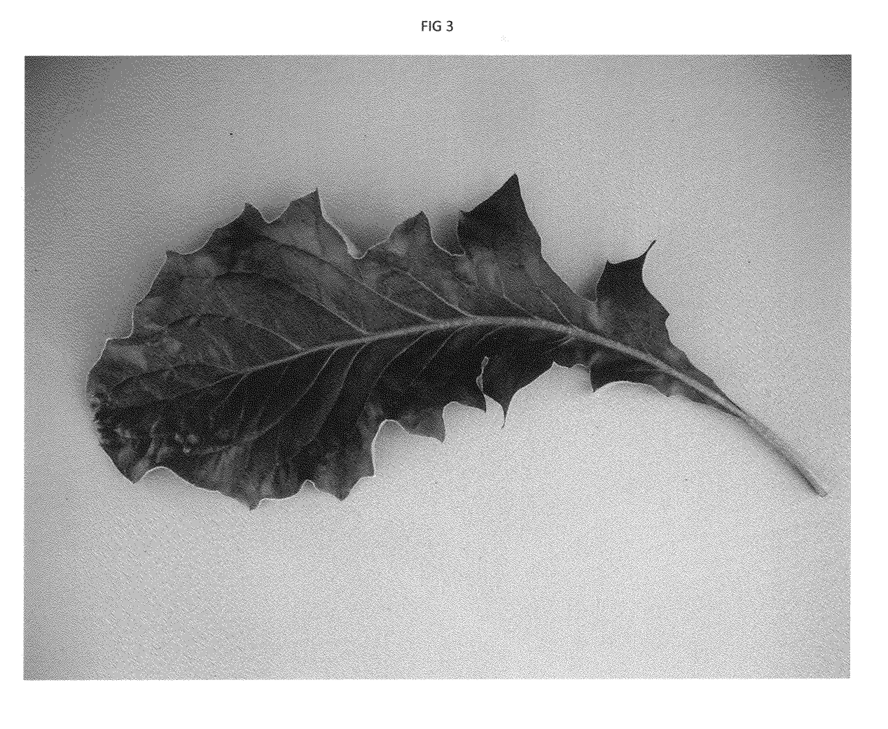

### DESCRIPTION OF THE NEW CULTIVAR

The following detailed description sets forth the distinctive characteristics of ‘UFGE 4141’. The present botanical description is that of the variety at approximately 5 months old on Oct. 20, 2009 at 3:30 p.m. in a room under north light in Wimauma, Fla. The colors (except those in common terms) are described from R.H.S. Colour Chart published by The Royal Horticultural Society in London (1986 ed.), in association with the Flower Council of Holland.

### DETAILED BOTANICAL DESCRIPTION

Classification:
   Botanical.—Gerbera hybrida cv. ‘UFGE 4141’.
   Common name.—Gerbera.
Parentage:
   Female parent.—‘UFGE 5-23’ (unpatented).
   Male parent.—‘UFGE 4-8’ (unpatented).
Inflorescence:
   Flower head.—Type: Semi-double Diameter: Large, approx. 120 mm Color (general tonality from a distance of 3 meters): Bright orange-red (RHS 33B) Shape: Incurving funnel-shaped.
   Involucre.—Height from point of attachment of involucre to top of flower head: Short, approx. 17 mm Diameter: Small to medium, approx. 43 mm Number of bracts: Medium, approx. 68 Color: Green (RHS 138A) Longitudinal axis of inner rows: Straight Anthocyanin: Absent Pubescence: Very dense.
   Inner ray florets.—Number: Medium, approx. 55 Shape: Narrow elliptic Longitudinal axis of outer row: Moderately incurving Longitudinal axis of inner row: Straight.
   Outer ray floret.—Cross section: Convex Length: Medium, approx. 56 mm Width: Medium, approx. 7 mm Shape of apex: Pointed Depth of incision: Shallow Color (topside): Orange-red (RHS 33B) Color (bottom side): Orange (RHS 25C) Color distribution on inner side: Uniform Edge of different color: None Striation: Absent.
   Disc florets.—Disc diameter: Medium, approx. 28 mm.
   Reproductive organs.—Style (main color of distal part): Orange (RHS 26B) Stigma (main color): Yellow (RHS 12B) Anthers (main color): Yellow-orange (RHS 17C) Color of top relative to other parts: Same Longitudinal stripes: Absent.
   Pappus.—Main color: Yellow-white (RHS 158D) Color of top relative to other parts: Same Level of top relative to closed disc florets: Above, 1-2 mm in center.
   Peduncle.—Length: Medium, approx. 47 cm Tendency to fasciation: Absent Thickness: Medium, approx. 6.5 mm Strength: Strong Pubescence: Dense Color: Yellow-green (RHS 144B) Anthocyanin coloration: At base: Very weak At top: Absent Involucral bracts: Absent.
Plant:
   General appearance.—Height: Approx. 52 cm, excluding any flowers Width: Approx. 65 cm.
   Foliage.—Leaf blade: Length: Long, approx. 26 cm Width: Medium, approx. 12 cm Thickness: Medium Blistering: Weak Pubescence (On upper side, midrib excluded): Sparse Depth of incisions in leaf: Basal part: Medium Central part: Shallow Distal part: Shallow Color: Upper side: Yellow-green (RHS 146A) Bottom side: Yellow-green (RHS 146B) Glossiness on upper side: Medium Shape of apex: Rounded Petiole: Petiole length: Medium, approx. 14 cm Color of petiole: Yellow-green (RHS 144B) Petiole anthocyanin coloration: Very weak.
Resistance to diseases: ‘UFGE 4141’ has been observed in multiple experiments in which it was compared with two common commercial cultivars, ‘Pensacola’ gerbera (unpatented) and ‘Bimini’ gerbera (unpatented), for the incidence and severity of powdery mildew, Podosphaera (syn. Sphaerotheca) fusca (Fr.) S. Blumer. ‘UFGE 4141’ has consistently been found to have a moderate level of resistance to powdery mildew. In two experiments conducted in 2008 and 2009 in Wimauma, Fla., tissue culture liners of ‘UFGE 4141’, ‘Bimini’, and ‘Pensacola’ were transplanted on into 2.7-L containers filled with commercial potting mix amended with controlled release fertilizer at the rate of 5.28 kg·m⁻³ and trace element fertilizer at the rate of 1.05 kg·m⁻³. Plants were grown under a plastic tunnel within a screen house with approximately 45% light exclusion. Fungicides were not applied throughout the experiments to subject the plants to natural powdery mildew disease pressures. In the first of the two powdery mildew experiments (Experiment 1), the severity of powdery mildew on gerbera leaves was assessed at week 8 after transplanting (Feb. 9, 2009), week 10 after transplanting (Feb. 18, 2009), week 12 after transplanting (Mar. 4, 2009), and week 14 after transplanting (Mar. 18, 2009). In the second of the two powdery mildew experiments (Experiment 2), the severity of powdery mildew was assessed at week 8 after transplanting (Oct. 15, 2009), week 10 after transplanting (Oct. 29, 2009), and week 12 after transplanting (Nov. 12, 2009). A randomized complete block design was used with five replications in Experiment 1 and with eight replications in Experiment 2. The experimental unit was a single containerized plant. ‘UFGE 4141’ showed a moderate level of resistance to powdery mildew in both Experiment 1 and Experiment 2 (Table 1). ‘UFGE 4141’ remained to be more productive than ‘Bimini’ and ‘Pensacola’ when natural powdery mildew disease pressure existed (Table 1). No other disease resistance characterizations have been made.

TABLE 1
Powdery mildew severity ratings and flower counts of ‘UFGE 4141’
and two commercial gerbera cultivars, ‘Bimini’ and
‘Pensacola’, grown under natural powdery mildew pressure
in two experimental trials in Wimauma, FL.
Experiment 1 (Winter 2008 through Spring 2009),
Experiment 2 (Summer 2009 through Fall 2009)

| | PM Ratings^{Z} | | | | Flower |
|---|---|---|---|---|---|
| Cultivars | Week 8 | Week 10 | Week 12 | Week 14 | counts^{Y} |
| | Experiment 1 (Winter 2008 through Spring 2009) | | | | |
| UFGE 4141 | 1.0 | 2.4 | 4.6 | 6.8 | 4.4 |
| Bimini | 1.2 | 1.8 | 5.4 | 8.0 | 3.2 |
| Pensacola | 1.0 | 2.0 | 4.8 | 8.6 | 3.0 |
| | Experiment 2 (Summer 2009 through Fall 2009) | | | | |
| UFGE 4141 | 1.1 | 3.5 | 5.3 | | 5.6 |
| Bimini | 2.6 | 8.6 | 9.4 | | 2.6 |
| Pensacola | 1.8 | 7.5 | 9.8 | | 1.1 |

^{Z}Powdery mildew severity was rated on a scale of 1 to 10 as described by Hausbeck et al. (2002): 1 = no disease, 2 = trace to 10%, 3 = 10% to 20%, 4 = 20 to 30%, 5 = 30% to 40%, 6 = 40% to 50%, 7 = 50% to 60%, 8 = 60% to 70%, 9 = 70% to 80%, and 10 = 80% to 100% of leaf surface covered with powdery mildew.
^{Y}Total number of flowers produced per plant over nine weeks from Jan. 20, 2009 to Mar. 17, 2009 in Experiment 1 or from Sep. 25, 2009 to Nov. 20, 2009 in Experiment 2, both in a screen house in Wimauma, FL.

### COMPARISON WITH PARENTAL AND KNOWN CULTIVARS

‘UFGE 4141’ differs from the female parent ‘UFGE 5-23’ (unpatented) by having much shorter peducles, many more leaves, and denser foliage under growing conditions in Wimauma, Fla. than ‘UFGE 5-23’. ‘UFGE 4141’ differs from the male parent ‘UFGE 4-8’ (unpatented) by having flowers that are red-orange and much larger under growing conditions in Wimauma, Fla. than ‘UFGE 4-8’.

Comparisons were made with the commercial gerbera variety ‘Bimini’ (unpatented) and the commercial gerbera variety ‘Pensacola’ (unpatented). Two experiments were conducted in a greenhouse at Wimauma, Fla. under standard greenhouse management practices to evaluate the plant performance (days to flower, flower quality, flower count, and plant quality) of ‘UFGE 4141’ for container plant production in comparison to ‘Bimini’ and ‘Pensacola’. In the first of the two plant performance experiments (Experiment 3), tissue culture liners were transplanted on Dec. 9, 2008 into 2.7-L containers filled with commercial potting mix amended with controlled release fertilizer at the rate of 5.28 kg·m⁻³ and trace element fertilizer at the rate of 1.05 kg·m⁻³. Potted plants were grown on metal benches in a glass house and spaced 46 cm×46 cm apart. Temperatures inside the greenhouse ranged from 18° C. to 33° C. Two hours of additional photoperiodic lighting were provided between Dec. 9, 2008 and Mar. 17, 2009. Fungicides and insecticides were applied as needed to control powdery mildew and western flower thrips. Flower quality was rated on a 1 to 5 scale: 1=very poor, 3=fair, some blemishes, but acceptable, and 5=excellent, bright, uniform, and no blemishes. Flower counts were recorded weekly from Jan. 20, 2009 through Mar. 17, 2009. Plant quality was rated three times, on Jan. 20, 2009, Feb. 3, 2009, and Feb. 17, 2009, using a 1 to 5 scale: 1=few leaves or long petioles, container surface visible, very poor and unacceptable as flowering pot plants, 3=fair and marketable, and 5=excellent, full, symmetrical, attractive plants.

In the second of the two plant performance experiments (Experiment 4), tissue culture liners were transplanted on Aug. 13, 2009 into 2.7-L plastic containers filled with commercial potting mix amended with controlled release fertilizer at the rate of 5.28 kg·m⁻³ and trace element fertilizer at the rate of 1.05 kg·m⁻³. Plants were grown on metal benches in a greenhouse with a spacing of 46 cm×46 cm. The greenhouse was covered with aluminum shading cloth with approximately 30% light exclusion. Greenhouse temperatures ranged from 21° C. to 35° C. Two hours of additional photoperiodic lighting was provided beginning Sep. 15, 2009. Fungicides and insecticides were applied as needed to control powdery mildew and western flower thrips. Flower quality and plant quality were rated as above for Experiment 3, but on Sep. 25, 2009, Oct. 15, 2009, and Nov. 5, 2009. The number of flowers produced per plant was recorded weekly from Sep. 25, 2009 through Nov. 20, 2009.

The experiment design for the plant performance experiments was a randomized complete block design with five replications in Experiment 3 and eight replications in Experiment 4. The experimental unit was a single containerized plant.

‘UFGE 4141’ came into flowering 26 to 31 days earlier than ‘Bimini’ and ‘Pensacola’ in Experiment 3 and 5 to 10 days earlier than ‘Bimini’ and ‘Pensacola’ in Experiment 4 (Table 2). Flower quality ratings of ‘UFGE 4141’ were 3.8 to 4.2, similar to that of ‘Pensacola’, but it was lower than that of ‘Bimini’ (Table 2). In both Experiment 3 and Experiment 4, ‘UFGE 4141’ produced more flowers (87% to 144% more than ‘Bimini’ and 300% more than ‘Pensacola’) (Table 2). The plant quality rating of ‘UFGE 4141’ was 3.7 and 4.4 in Experiment 3 and Experiment 4, respectively, higher than that of the controls (Table 2).

TABLE 2
Plant performance of ‘UFGE 4141’ and two commercial cultivars,
‘Bimini’ and ‘Pensacola’ grown in Experiment 3
(Winter 2008 through Spring 2009) and Experiment 4
(Summer 2009 through Fall 2009) in Wimauma, Florida.

| | Days to 1st open | Flower | Flower counts^{Z} | Plant |
|---|---|---|---|---|
| Cultivars | flowers (no.) | quality^{Z} | (no.) | quality^{Y} |
| | Experiment 3 (Dec. 9, 2008 to Mar. 17, 2009) | | | |
| UFGE 4141 | 37.8 | 3.8 | 5.6 | 3.7 |
| Bimini | 64.0 | 5.0 | 3.0 | 3.1 |
| Pensacola | 68.8 | 4.1 | 1.4 | 3.3 |
| | Experiment 4 (Aug. 13, 2009 to Nov. 20, 2009) | | | |
| UFGE 4141 | 44.6 | 4.2 | 11.9 | 4.4 |
| Bimini | 50.1 | 4.8 | 5.5 | 3.2 |
| Pensacola | 55.8 | 4.3 | 2.5 | 3.3 |

^{Z}Flower count data were collected over nine weeks from Jan. 20, 2009 to Mar. 17, 2009 in Experiment 3 and another nine weeks from Sep. 25, 2009 to Nov. 20, 2009 in Experiment 4. Each value represents the mean of five (Experiment 3) or eight (Experiment 4) replicates.
^{Y}The plant quality rating value in Experiment 3 was the mean of five replicates over three times (Jan. 20, Feb. 3, and Feb. 17, 2009), and the plant quality rating value in Experiment 4 was the mean of eight replicates over three times (Sep. 25, Oct. 15, and Nov. 5, 2009).

### Literature Cited

Hausbeck, M. K., W. R. Quackenbush, and S. D. Linderman. 2002. Evaluation of cultivars of African daisy for resistance to powdery mildew, 2002. B&C Tests 18:O0004.

I claim:

1. A new and distinct cultivar of Gerbera plant named ‘UFGE 4141’, as illustrated and described herein.
